(19) United States
(12) Patent Application Publication
(10) Pub. No.: US 2026/0048119 A1
(43) Pub. Date: Feb. 19, 2026

(54) ANTI-CTLA4 ANTIBODIES FOR TREATMENT OF KRAS MUTANT CANCERS

(71) Applicant: BRISTOL-MYERS SQUIBB COMPANY, Princeton, NJ (US)

(72) Inventors: David Edward BALLI, Warrington, PA (US); Wendy CLEMENS, PERKASIE, PA (US); William J. GEESE, PIPERSVILLE, PA (US); Amy HAMMELL-MCLAUGHLIN, Hillsborough, NJ (US)

(73) Assignee: BRISTOL-MYERS SQUIBB COMPANY, Princeton, NJ (US)

(21) Appl. No.: 19/101,411

(22) PCT Filed: Aug. 4, 2023
(86) PCT No.: PCT/US2023/071636

§ 371 Date: Feb. 5, 2025

Related U.S. Application Data
(60) Provisional application No. 63/370,608, filed on Aug. 5, 2022.

(51) Int. Cl. A61K 39/395; A61K 39/00; A61P 35/00; C07K 16/24; C07K 16/28; C12Q 1/6886; G01N 33/574

(52) U.S. Cl. CPC A61K 39/39541; A61P 35/00; C07K 16/244; C07K 16/2827; C12Q 1/6886; G01N 33/57492; A61K 2039/507; C07K 2317/41; C07K 2317/732

(57) ABSTRACT

The present invention provides methods of treating cancer patients having KRAS mutant tumors, and methods of selecting such patients for treatment, comprising selectively administering an anti-CTLA-4 antibody to those patients having KRAS mutations. Exemplary anti-CTLA-4 antibodies for use in the methods of the invention include ipilimumab, nonfucosylated anti-CTLA-4 antibodies, activatable anti-CTLA-4 antibodies, and nonfucosylated activatable anti-CTLA-4 antibodies. Such methods optionally comprise concurrent treatment with an anti-PD-1 or anti-PD-L1 antibody.

### CROSS REFERENCE TO RELATED APPLICATIONS

This application claims priority to U.S. Provisional Application No. 63/370,608, filed 5 Aug. 2022, the disclosure of which is incorporated herein by reference.

### SEQUENCE LISTING

The Sequence Listing filed electronically herewith is also hereby incorporated by reference in its entirety (File Name: 20230727_SEQL_14264WOPCT_GB.xml; Date Created: 27 Jul. 2023; File Size: 35 KB).

### FIELD OF THE INVENTION

The present application discloses methods of treating cancer patients having KRAS mutant tumors comprising administration of an anti-CTLA-4 antibody, including certain variants thereof, optionally along with other therapy such as anti-PD-1/PD-L1 antibody, as well as methods of selecting patients for anti-CTLA-4 treatment based on KRAS mutation status.

### BACKGROUND OF THE INVENTION

The immune system is capable of controlling tumor development and mediating tumor regression. This requires the generation and activation of tumor antigen-specific T cells. Multiple T-cell co-stimulatory receptors and T-cell negative regulators, or co-inhibitory receptors, act in concert to control T-cell activation, proliferation, and gain or loss of effector function. Among the earliest and best characterized T-cell co-stimulatory and co-inhibitory molecules are CD28 and CTLA-4. Rudd et al. (2009) Immunol. Rev. 229:12. CD28 provides co-stimulatory signals to T-cell receptor engagement by binding to B7-1 and B7-2 ligands on antigen-presenting cells, while CTLA-4 provides a negative 30 signal, down-regulating T-cell proliferation and function. CTLA-4, which also binds the B7-1 (CD80) and B7-2 (CD86) ligands but with higher affinity than CD28, acts as a negative regulator of T-cell function through both cell autonomous (or intrinsic) and cell non-autonomous (or extrinsic) pathways. Intrinsic control of CD8 and CD4 T effector (T_{eff}) function is mediated by the inducible surface expression of CTLA-4 as a result of T-cell activation, and inhibition of T-cell proliferation by multivalent engagement of B7 ligands on opposing cells. Peggs et al. (2008) Immunol. Rev. 224:141.

Anti-CTLA-4 antibodies, when cross-linked, suppress T cell function in vitro. Krummel & Allison (1995) J. Exp. Med 182:459: Walunas et al. (1994) Immunity 1:405. Regulatory T cells (T_{regs}), which express CTLA-4 constitutively, control T_{eff} function in a non-cell autonomous fashion. T_{regs} that are deficient for CTLA-4 have impaired suppressive ability (Wing et al. (2008) Science 322:271) and antibodies that block CTLA-4 interaction with B7 can inhibit T_{reg} function (Read et al. (2000) J. Exp. Med 192:295: Quezada et al. (2006) J. (Cin. Invest. 116:1935). More recently, Tens have also been shown to control T cell function through extrinsic pathways (Corse & Allison (2012) J. Immunol. 189:1123; Wang et al. (2012) J. Immunol. 189:1118). Extrinsic control of T cell function by T_{regs} and T_{effs} occurs through the ability of CTLA-4-positive cells to remove B7 ligands on antigen-presenting cells, thereby limiting their co-stimulatory potential. Qureshi et al. (2011) Science 332: 600; Onishi et al. (2008) Proc. Nat'l Acad. Sci. (USA) 105:10113. Antibody blockade of CTLA-4/B7 interactions is thought to promote T_{eff} activation by interfering with negative signals transmitted by CTLA-4 engagement; this intrinsic control of T-cell activation and proliferation can promote both T_{eff} and T_{reg} proliferation (Krummel & Allison (1995) J. Exp. Med 182:459; Quezada et al. (2006) J. Clin. Invest. 116:1935). In early studies with animal models, antibody blockade of CTLA-4 was shown to exacerbate autoimmunity. Perrin et al. (1996) J. Immunol. 157:1333: Hurwitz et al. (1997) J. Neuroimmunol. 73:57. By extension to tumor immunity, the ability of anti-CTLA-4 to cause regression of established tumors provided a dramatic example of the therapeutic potential of CTLA-4 blockade. Leach et al. (1996) Science 271:1734.

Human antibodies to human CTLA-4, ipilimumab and tremelimumab, were selected to inhibit CTLA-4-B7 interactions (Keler et al. (2003) J. Immunol. 171:6251; Ribas et al. (2007) Oncologist 12:873) and have been tested in a variety of clinical trials for multiple malignancies. Hoos et al. (2010) Semin. Oncol. 37:533; Ascierto et al. (2011) J. Transl. Med. 9:196. Ipilimumab, which was first approved for the treatment of metastatic melanoma, has since been approved for use in other cancers. Hoos et al. (2010) Semin. Oncol. 37:533; Hodi et al. (2010) N. Engl. J. Med 363:711; Pardoll (2012) Nat. Immunol. 13(12):1129. In 2011, ipilimumab, which has an IgG1 constant region, was approved in the US and EU for the treatment of unresectable or metastatic melanoma based on an improvement in overall survival in a phase III trial of previously treated patients with advanced melanoma. Hodi et al. (2010) N. Engl. J. Med. 363:711. Tumor regressions and disease stabilization were frequently observed, but treatment with these antibodies was accompanied by adverse events with inflammatory infiltrates capable of affecting a variety of organ systems. The severity and frequency of side effects from treatment with ipilimumab, which carries a black box warning of immune-mediated adverse reactions, and to an even greater extent when combined with nivolumab (OPDIVO®), limits the use of ipilimumab by many treating physicians.

Next generation anti-CTLA-4 antibodies are being developed with improved properties. Activatable forms of ipilimumab have been developed in which the light chain contains a masking moiety that interferes with binding to CTLA-4, but is preferentially released in the tumor microenvironment after cleavage by proteases that are more prevalent and/or active in tumors than in peripheral tissues. WO 18/085555. Such preferential cleavage in the tumor microenvironment enables full CTLA-4 blocking, promoting anti-tumor immune response, while minimizing CTLA-4 blockade in normal tissue, where it would otherwise cause systemic toxicity. As a consequence, the activatable form exhibits an increased therapeutic index compared with the native parent molecule. One such activatable anti-CTLA-4 antibody has entered human clinical trials (NCT03369223: “A Study of BMS-986249 Alone and in Combination with Nivolumab in Advanced Solid Tumors”).

Anti-CTLA-4 antibodies with enhanced Fcγ receptor (CD16) binding, such as non-fucosylated anti-CTLA-4 antibodies, have also been proposed as therapeutic agents for treatment of cancer through depletion of T_{regs}. WO 14/089113. One such nonfucosylated anti-CTLA-4 antibody has entered human clinical trials (e.g., NCT03110107: “First-In-Human Study of Monoclonal Antibody BMS-986218 by Itself and in Combination with Nivolumab in Participants with Advanced Solid Tumors”). BMS-986218 is a non-fucosylated antibody developed to increase the effects of CTLA-4 blockade by enhancing binding to Fcy receptor, thus promoting APC-mediated T cell priming. Activatable anti-CTLA-4 antibodies can also be produced as nonfucosylated antibodies having both the increased efficacy of an enhanced Fcy receptor binding antibody, with the added safety of being masked in peripheral tissues. One such nonfucosylated activatable anti-CTLA-4 antibody has entered human clinical trials (NCT03994601: “An Investigational Immunotherapy Study of BMS-986288 Alone and in Combination with Nivolumab in Advanced Solid Cancers”).

Although anti-CTLA-4 antibodies, including next generation CTLA-4 antibodies, optionally combined with anti-PD1/PD-L1 antibody therapy, provide therapeutic benefits, such benefits are not observed in all patients. The need exists for methods of treating patients who are most likely to experience clinical benefit by treatment with anti-CTLA-4 antibodies, including next generation anti-CTLA-4 antibodies, and methods of selecting the patients most likely to experience clinical benefit by treatment with anti-CTLA-4 antibodies, including next generation anti-CTLA-4 antibodies.

### SUMMARY OF THE INVENTION

The present invention provides methods of selectively treating cancer patients having KRAS mutations in their tumors comprising administering anti-CTLA-4 antibodies, including next generation anti-CTLA-4 antibodies. The invention further provides methods of treatment of cancer patients comprising i) determining which patients have KRAS mutations in their tumors, and ii) selectively administering anti-CTLA-4 antibodies, including next generation anti-CTLA-4 antibodies, to the patients that have KRAS mutations in their tumors and not to patients that do not have KRAS mutations in their tumors. The invention further provides methods of selecting cancer patients who will show the greatest benefit from treatment with anti-CTLA-4 antibodies, including next generation anti-CTLA-4 antibodies, based on the presence of mutations in the KRAS gene in their tumors, wherein patients having KRAS mutant tumors are selected for treatment with anti-CTLA-4 antibodies and patients having wild-type KRAS tumors are not. Such methods of treatment and methods of patient selection comprise means for detecting KRAS mutations in patient tumors.

In some embodiments the patients being treated or selected for treatment with the methods of the present invention are treated with combination therapy of anti-CTLA-4 antibodies, including next generation anti-CTLA-4 antibodies, and anti-PD1 or anti-PD-L1 antibodies, such as nivolumab, pembrolizumab, cemiplimab, durvalumab, avelumab, or atezolizumab.

In some embodiments patients being treated or selected for treatment with the methods of the present invention have previously been treated with anti-PD1 and/or anti-PD-L 1 antibodies without anti-CTLA-4 treatment. In various embodiments this prior treatment was not entirely effective in treating the patient's cancer, or the patient has suffered a relapse.

In some embodiments, the anti-CTLA-4 antibody for use in the methods of treatment and medical uses herein comprises the CDRH1, CDRH2, CDRH3, CDRL1, CDRL2 and CDRL3 sequences of SEQ ID NOs: 3-8, respectively. In another embodiment, the anti-CTLA-4 antibody comprises the variable heavy chain (V_{H}) and variable light chain (V_{L}) sequences of SEQ ID NOs: 9 and 13, respectively. In a further embodiment, the anti-CTLA-4 antibody comprises the heavy chain (HC) sequence of SEQ ID NO: 11 or 12, and the light chain (LC) sequence of SEQ ID NO: 15.

In a further alternative embodiment, the anti-CTLA-4 antibody is an activatable anti-CTLA-4 antibody, such as an anti-CTLA-4 antibody comprising the V_{H} and V_{L} sequences of SEQ ID NOs: 9 and 22, respectively. In a further embodiment, the activatable anti-CTLA-4 antibody comprises the HC sequence of SEQ ID NO: 11 or 12, and the LC sequence of SEQ ID NO: 24.

In another embodiment, the anti-CTLA-4 antibody or activatable anti-CTLA-4 antibody of the preceding two paragraphs has enhanced Fcy receptor binding. Enhancement of Fcy receptor binding is defined by comparison with the Fcy receptor binding of ipilimumab, which for purposes of this discussion does not have enhanced Fcy receptor binding. In some embodiments, the anti-CTLA-4 antibody (or activatable anti-CTLA-4 antibody) with enhanced Fcy receptor binding lacks fucose residues in its N-linked glycans, i.e. it is nonfucosylated (NF). In some embodiments the nonfucosylated anti-CTLA-4 antibody (or nonfucosylated activatable anti-CTLA-4 antibody) is produced by expressing the chains of the antibody in a mammalian cell under conditions that prevent fucosylation, including but not limited to use of mammalian cells with genetic modifications preventing fucosylation, or growth of the cells expressing the antibody in medium containing one or more chemical compounds that inhibit fucosylation. In one embodiment, the genetic modification that prevents fucosylation is inactivation, e.g. knock-out, of the FUT8 gene. In other embodiments of an anti-CTLA-4 antibody with enhanced Fcy receptor binding, the Fc region of the anti-CTLA-4 antibody contains amino acid substitutions to enhance binding to activating Fcy receptors.

In various embodiments the patients being treated or selected for treatment using the methods of the present invention have non-small cell lung cancer (NSCLC). In some embodiments the NSCLC is non-squamous (NSQ) NSCLC.

In some embodiments the patients being treated or selected for treatment using the methods of the present invention have tumors with a KRAS mutation. In some embodiments the patients being treated or selected for treatment using the methods of the present invention have tumors with a G12C KRAS mutation. Methods of treatment and methods of patient selection of the present invention comprise means for detecting KRAS mutations in patient tumors, optionally including specific detection of the G12C mutation.

In selected embodiments the patients being treated or selected for treatment using the methods of the present invention have tumors with PD-L1 expression in 21% of tumor cells, that is they have a Tumor Proportion Score (TPS) of 2%.

### BRIEF DESCRIPTION OF THE DRAWINGS

FIGS. 1A and 1B show, respectively, the probability of progression free survival (PFS) and overall survival (OS), for NSCLC patients (NSQ, PD-L1 ≥1%) treated with chemotherapy (“CHEMO”—ARM C), nivolumab (“NIVO”—ARM A), or a combination of nivolumab and ipilimumab (“NIVO+IPI”—ARM B) as a function of the number of months on treatment. See Example 1 (Part 1). Plots show results for NIVO+IPI (upper curve at 36 months), NIVO (middle curve at 36 months) and CHEMO (lower curve at 36 months). Reference to data at 36 months, and at other specific times in other figures, is provided only for ease of distinguishing curves from one another, and is of no other significance. Numerical values for median PFS or median OS (measured in months, in this and all other figures herein) are also provided. The results show that for all patients, not segregated by KRAS mutation status, there is a benefit to treatment with regimens including ipilimumab.

FIGS. 2A and 2B show the PFS data from FIG. 1A broken out by KRAS mutant status, with data from patients with KRAS mutant tumors at FIG. 2A and data from patients with KRAS wild-type (WT) tumors at FIG. 2B. Upper, middle and lower curves are as described for FIGS. 1A and 1B, with curve separation clearest at 18 months rather than 36 months. Numerical values for median PFS are also provided. The data show that patients with KRAS mutant tumors benefit more from addition of ipilimumab to nivolumab than patients with WT KRAS tumors.

FIG. 3A shows the PFS data from FIG. 2A for patients having the G12C mutation in KRAS, rather than any/all mutations in KRAS. FIG. 3B shows the median PFS data from FIGS. 2A and 2B for patients having either WT KRAS or a mutation other than G12C. Upper, middle and lower curves are as described for FIGS. 1A and 1B, with curve separation clearest at 12 months rather than 36 months. Numerical values for median PFS are also provided. The data show that patients with the G12C mutation in KRAS benefit more from addition of ipilimumab to nivolumab than the combination of patients with WT KRAS tumors and non-G12C KRAS mutations.

FIGS. 4A and 4B show the probability of OS for NSCLC patients (NSQ, PD-L1 ≥1%) treated with chemotherapy (“CHEMO”—ARM C), nivolumab (“NIVO”—ARM A), or a combination of nivolumab and ipilimumab (“NIVO+IPI”—ARM B) as a function of the number of months on treatment, broken out by patients with mutant KRAS tumors (FIG. 4A) and WT KRAS tumors (FIG. 4B). See Example 1 (Part 1). For FIG. 4A, results are provided for NIVO+IPI (upper curve at 15 months), CHEMO (middle curve at 15 months), and NIVO (lower curve at 15 months). For FIG. 4B, results are provided for NIVO+IPI (upper curve at 57 months), and for NIVO (middle curve at 57 months) and CHEMO (lower curve at 57 months), which are substantially overlapping at most times. Numerical values for median OS are also provided. The results show that patients with KRAS mutant tumors benefit more from addition of ipilimumab to their treatment regimen than patients with WT KRAS tumors.

FIG. 5 shows percent ORR in NSCLC patients (NSQ, PD-L1 ≥1%) treated with NIVO, NIVO+IPI or CHEMO, comparing KRAS mutant (gray, right bars) compared to KRAS WT (dark, left bars) patients. See Example 1 (Part 1). Patient numbers are provided above the bars. While patients having KRAS mutant tumors fare worse than WT KRAS patients when treated with chemotherapy or nivolumab alone, they fare better than patients with WT KRAS tumors when ipilimumab is included with nivolumab treatment. Viewed another way, addition of ipilimumab is selectively beneficial to patients having KRAS mutant tumors.

FIG. 6A shows the probability of OS data from FIG. 4A for patients having the G12C mutation in KRAS, rather than any/all mutations in KRAS. FIG. 6B shows the probability of OS for all other patients, both WT and KRAS mutants other than G12C. Upper, middle and lower curves for FIG. 6A are the same as for FIG. 4A, but with curve separation clearest at 18 months rather than 15 months. Curves for NIVO and CHEMO arms in FIG. 6B are substantially overlapping, with the curve for NIVO+IPI being the upper curve at 54 months. Numerical values for median OS are also provided. The results show that patients with the G12C mutation in KRAS benefit more from addition of ipilimumab to nivolumab than the combination of patients with WT KRAS tumors and non-G12C KRAS mutations.

FIGS. 7A and 7B show the probability of PFS for NSCLC patients (NSQ, PD-L1 ≥1%) treated with chemotherapy (“CHEMO”) or a combination of nivolumab, ipilimumab and chemo (“NIVO+IPI+CHEMO”) as a function of the number of months on treatment, for patients having KRAS mutant tumors (FIG. 7A) and for patients having WT KRAS tumors (FIG. 7B). See Example 2. NIVO+IPI+CHEMO is the upper curve in FIG. 7A, and is substantially overlapping the curve for CHEMO in FIG. 7B. Numerical values and statistical values are provided in tabular form. Numerical values for median PFS are also provided. The results show that addition of nivolumab and ipilimumab to chemotherapy significantly improves overall survival.

### DETAILED DESCRIPTION OF THE INVENTION

### Definitions

In order that the present disclosure may be more readily understood, certain terms are first defined. As used in this application, except as otherwise expressly provided herein, each of the following terms shall have the meaning set forth below. Additional definitions are set forth throughout the application.

“Activatable antibodies,” as used herein, refers to modified forms of antibodies that bind to targets of therapeutic interest wherein the antibodies comprise structural modifications that inhibit binding to the target until cleaved by proteases more prevalent and/or active in the tumor microenvironment than in peripheral tissue. “Activatable antibodies” encompasses activatable forms of anti-CTLA-4 antibody ipilimumab, such as antibodies comprising light chains modified to comprise a masking moiety (MM) and a cleavable moiety (CM), as disclosed in WO 18/085555, for example, Activatable Ipilimumab.

“Activatable Ipilimumab,” as used herein, refers to an activatable form of ipilimumab comprising a heavy chain comprising the heavy chain variable region sequence of SEQ ID NO: 9 and a light chain comprising a light chain variable region sequence selected from the group consisting of SEQ ID NOs: 21, 22 and 23. The light chain variable domain of an Activatable Ipilimumab may optionally further comprise a spacer of SEQ ID NO: 16 and the light chain may comprise a kappa constant domain of SEQ ID NO: 14, for example the spacer YV39-2011 light chain provided at SEQ ID NO: 24. The heavy chain of an Activatable Ipilimumab may further comprise an IgG1 constant domain of SEQ ID NO: 10, for example as in the ipilimumab heavy chain provided at SEQ ID NO: 11 or 12. Activatable Ipilimumab may comprise a heavy chain comprising SEQ ID NO: 11 or 12 and a light chain comprising a light chain of SEQ ID NO. 24, and may be BMS-986249. An Activatable Ipilimumab with enhanced Fcy receptor binding is an Activatable Ipilimumab modified to enhance Fcy receptor binding, for example produced without fucose in its glycan, for example produced in a cell line lacking FUT8 activity, and may be BMS-986288.

“Administering,” “administer” or “administration” refers to the physical introduction of a composition comprising a therapeutic agent to a subject, using any of the various methods and delivery systems known to those skilled in the art. Preferred routes of administration for antibodies of the invention include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody of the invention can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

Administering also includes prescribing a medication, even if the medication is actually delivered by another medical professional, or by the patient himself or herself.

Unless otherwise indicated, administration of antibodies for the treatment of cancer is parenteral, such as intravenous (iv) or subcutaneous (sc). Methods of dosing and administration of the present invention can be performed for any number of cycles of treatment, from one, two, three, four cycles, etc., up to continuous treatment (repeating the dosing until no longer necessary, disease recurrence, or unacceptable toxicity is reached). For combination therapy, one cycle comprises the minimal unit of administration that includes at least one dose of each component (drug) of the combination therapy.

“Approximately,” as used herein with respect to amounts and concentrations of components of the various formulations herein, refers to ranges of values typically obtained in pharmaceutical formulations, such as amounts and concentrations within manufacturing tolerances. The degree of batch-to-batch variation that is considered within tolerances of the desired numerical (“nominal”) amount or concentration defines what is “approximately” the nominal amount or concentration.

“Initial Dose” or “initial dosing” as used herein refers to the first dosing of a patient with the regimen, and any subsequent repetitions of that same dosing regimen (such as second, third and fourth cycles, etc.), and is contrasted with “maintenance dose” or “maintenance dosing,” which refers to subsequent doses administered over a longer period after the initial dose or doses, e.g. longer than three months up to several years, or even indefinitely. Maintenance dosing may optionally comprise less frequent dosing and/or lower dose than the initial dose.

“Combination therapy,” as used herein, refers to administration of two or more therapeutic agents in a coordinated treatment plan, in which the dose and dosing interval of a first component of the combination is based on the dose and dosing interval of a second component, to elicit an overall therapeutic benefit. It is not limited to any particular details of administration, and encompasses administration as a mixture of the components, administration as separate compositions, whether concurrent or sequential on a given day. Although combination therapy is most convenient when dosing schedules are the same or multiples of one another (e.g. Q4W and Q8W), it also encompasses administration on different days if dosing intervals do not align for any given cycle.

An “antibody” (Ab) shall include, without limitation, a glycoprotein immunoglobulin or immunoglobulin which binds specifically to an antigen and comprises at least two heavy chains (HC) and two light chains (LC) interconnected by disulfide bonds. Each heavy chain comprises a heavy chain variable region (abbreviated herein as V_{H}) and a heavy chain constant region. The heavy chain constant region comprises three domains, C_{H1}, C_{H2} and C_{H3}. Each light chain comprises a light chain variable region (abbreviated herein as Vt.) and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved between antibodies, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains comprise a binding domain that interacts with an antigen.

As used herein, and in accord with conventional interpretation, an antibody that is described as comprising “a” heavy chain and/or “a” light chain refers to antibodies that comprise “at least one” of the recited heavy and/or light chains, and thus will encompass antibodies having two or more heavy and/or light chains. Specifically, antibodies so described will encompass conventional antibodies having two substantially identical heavy chains and two substantially identical light chains. Such antibodies also include bispecific antibodies comprising, e.g., two distinct heavy chains and two light chains, which can be distinct from each other or can be identical (a common light chain bispecific mAb). Antibody chains may be substantially identical but not entirely identical if they differ due to post-translational modifications, such as C-terminal cleavage of lysine residues, alternative glycosylation patterns, etc. Antibodies differing in fucosylation within the glycan, however, are not substantially identical.

When used with reference to activatable antibodies, the “light chain variable domain” may further comprise a masking moiety, a cleavable moiety, a spacer element and optionally other sequence elements as disclosed herein.

Unless otherwise indicated or clear from the context, an antibody defined by its target specificity (e.g. an “anti-CTLA-4 antibody”) refers to an antibody that can bind to its human target (i.e. human CTLA-4). Such antibodies may or may not bind to CTLA-4 from other species.

The immunoglobulin may derive from any of the commonly known isotypes, including but not limited to IgA, secretory IgA, IgG and IgM. The IgG isotype may be divided in subclasses in certain species: IgG1, IgG2, IgG3 and IgG4 in humans, and IgG1, IgG2a, IgG2b and IgG3 in mice. “Isotype” refers to the antibody class (e.g., IgM or IgG1) that is encoded by the heavy chain constant region genes. “Antibody” includes, by way of example, both naturally occurring and non-naturally occurring antibodies, including allotypic variants; monoclonal and polyclonal antibodies; chimeric and humanized antibodies; human or non-human antibodies: wholly synthetic antibodies; and single chain antibodies. Unless otherwise indicated, or clear from the context, antibodies disclosed herein are human IgG1 antibodies. IgG1 constant domain sequences include, but are not limited to, known IgG1 allotypic variants.

The term “monoclonal antibody” (“mAb”) refers to a preparation of antibody molecules of single molecular composition, i.e., antibody molecules whose primary amino acid sequences are identical or essentially identical, and which exhibit a single binding specificity and affinity for a particular epitope. Monoclonal antibodies may be produced by hybridoma, recombinant means, transgenic animals or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies are used synonymously.

An “antibody fragment” refers to a portion of a whole antibody, generally including the “antigen-binding portion” (“antigen-binding fragment”) of an intact antibody which retains the ability to bind specifically to the antigen bound by the intact antibody.

“Antibody-dependent cell-mediated cytotoxicity” or “antibody-dependent cellular cytotoxicity” (ADCC) refers to an in vitro or in vivo cell-mediated reaction in which nonspecific cytotoxic cells that express FcRs (e.g., natural killer (NK) cells, macrophages, neutrophils and eosinophils) recognize antibody bound to a surface antigen on a target cell and subsequently cause lysis of the target cell. In principle, any effector cell with an activating FcR can be triggered to mediate ADCC.

“Cancer” refers to a broad group of various diseases characterized by the uncontrolled growth of abnormal cells in the body. Unregulated cell division and growth results in the formation of malignant tumors or cells that invade neighboring tissues and may also metastasize to distant parts of the body through the lymphatic system or bloodstream.

A “cell surface receptor” refers to molecules and complexes of molecules capable of receiving a signal and transmitting such a signal across the plasma membrane of a cell.

“Detecting, or ordering the detection of,” as used herein with reference to determination of the KRAS mutation status of a tumor from a patient, refers to either the act of performing a method of detecting such mutations, e.g. as described in Example 5, or the act of instructing, ordering or directing that such method of detecting be performed by others. Such instructing, ordering or directing might be performed, for example, by a physician, or another medical professional under the direction of a physician, and might involve ordering a test to be performed at a commercial laboratory or in a medical facility laboratory. Such method of detection might also be performed in the physician's office using specialized equipment, either by the physician or another under the physician's direction. The physician who is responsible for instructing, ordering or directing such testing would typically also be the one to prescribe and/or administer the anti-CTLA-4 antibody to those patients with KRAS mutations.

“Effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and down-regulation of a cell surface receptor (e.g., the B cell receptor: BCR). Such effector functions generally require the Fc region to be combined with a binding domain (e.g., an antibody variable domain).

“Enhanced Fcy receptor binding” as used herein with reference to the anti-CTLA-4 antibodies of the present invention, refers to Fcy receptor binding levels greater than Fcy receptor binding of unmodified ipilimumab. Ipilimumab with enhanced Fcy receptor binding of the present invention, for example, is a modified form of ipilimumab that induces greater Fcy receptor binding than ipilimumab with its native IgG1 constant domain. As used herein, unless otherwise indicated or clear from the context, “Fcy receptor” refers to the activating receptor Fcy RIIIa (CD16). The level of enhancement in Fcy receptor binding may be measured as at least a two-fold, and optionally at least a ten-fold, reduction in the EC₅₀ for NK92 cell mediated cell lysis in the ADCC assay described at Example 3.

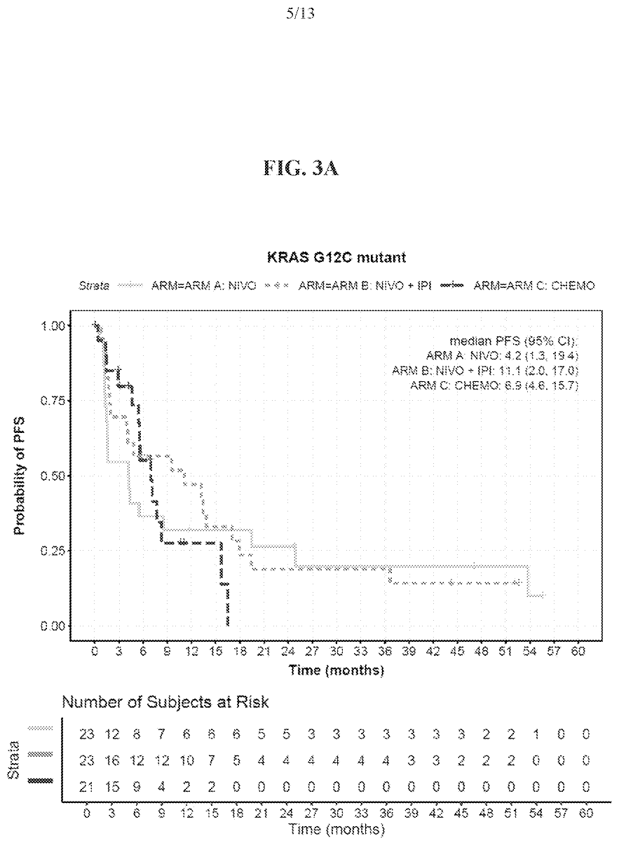

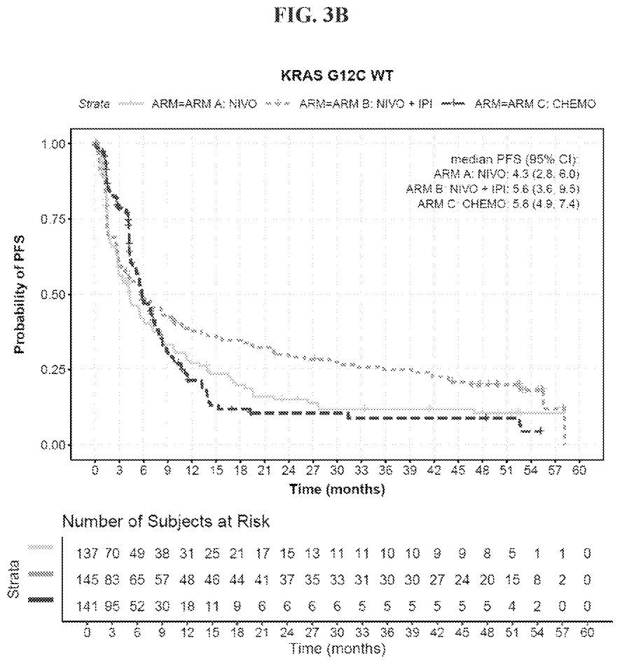

“Fucosylation” and “nonfucosylation.” as used herein, refer to the presence or absence of a core fucose residue on the N-linked glycan at position N297 of an antibody (EU numbering). Unless otherwise indicated, or clear from the context, amino acid residue numbering in the Fc region of an antibody is according to the EU numbering convention (the EU index as in Kabat et al. (1991) Sequences of Proteins of Immunological Interest. National Institutes of Health, Bethesda. MD; see also FIGS. 3c-3f of U.S. Pat. App. Pub. No. 2008/0248028, except when specifically referring to residues in a sequence in the Sequence Listing, in which case numbering is necessarily consecutive. For example, literature references regarding the effects of amino acid substitutions in the Fc region will typically use EU numbering, which allows for reference to any given residue in the Fc region of an antibody by the same number regardless of the length of the variable domain to which it is attached. In rare cases it may be necessary to refer to the document being referenced to confirm the precise Fc residue being referred to.

The phrase “if and only if” as used herein, is intended to act as a logical operator. For example, at patient that is treated with an anti-CTLA-antibody “if and only if” they have a KRAS mutant tumor is treated with the anti-CTLA-antibody if they have the KRAS mutant tumor but is not treated with the anti-CTL A-antibody if they do not have a KRAS mutant tumor.

An “immune response” refers to a biological response within a vertebrate against foreign agents, which response protects the organism against these agents and diseases caused by them. The immune response is mediated by the action of a cell of the immune system (for example, a T lymphocyte. B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues.

An “immunomodulator” or “immunoregulator” refers to a component of a signaling pathway that may be involved in modulating, regulating, or modifying an immune response. “Modulating,” “regulating,” or “modifying” an immune response refers to any alteration in a cell of the immune system or in the activity of such cell. Such modulation includes stimulation or suppression of the immune system which may be manifested by an increase or decrease in the number of various cell types, an increase or decrease in the activity of these cells, or any other changes that can occur within the immune system. Both inhibitory and stimulatory immunomodulators have been identified, some of which may have enhanced function in a tumor microenvironment. In preferred embodiments of the disclosed invention, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying an immune response.

“Ipilimumab,” “ipi” or YERVOY®, as used herein in the specification and figures, unless otherwise expressly indicated, refer to the antibody comprising the light chain of SEQ ID NO: 15 and the heavy chain of SEQ ID NO: 11 (lacking C-terminal lysine residue).

“KRAS,” as used herein, refers to the human Kirsten rat sarcoma virus oncogene. KRAS, as used herein, may refer to the genetic locus or the gene product, depending on the context. A “mutation” or “mutant” KRAS tumor is a tumor in a human that has one or more mutations (e.g. amino acid substitutions, deletions, insertions, frame-shifts, nonsense or gene amplification) compared to the wild-type human KRAS sequence and is associated with cancer. A KRAS mutation does not encompass KRAS having the wild type human KRAS sequence, and does not encompass sporadic mutations in KRAS that may be found in individual patients that are not known to be associated with cancer. Exemplary mutations in KRAS in NSCLC include mutations at codons 12 and 13, such as G12C, G12C, G12V. G12D, G12A, G12S, G12R, G12F, G13C, G13D, Q61L, Q61H, Q61K, A146T, and L19F. The most common mutations in NSCLC are G12C, G12V and G12D. Karachaliou et al. (2013) Clin. Lung Cancer 14:205. The presence or absence of mutations in KRAS in tumors of cancer patients may be detected by any method known in the art. Exemplary methods, which comprise means for detecting KRAS mutations, are provided at Example 5 herein.

“Means for detecting KRAS mutations” refers to any method known in the art or suitable for detection of genetic mutations in the DNA of tumor cells in a patient, including the methods provided in Example 5 and equivalents.

“Means for measuring Tumor Prognosis Score (TPS)” or “means for measuring the percentage of tumor cells expressing PD-L1” refer to any method known in the art or suitable for detection of the level of tumor cells expressing PD-L1, e.g., suitable for detecting which patients have tumors with ≥1% of cells expressing PD-L1. Such means include the methods provided in Example 6 and equivalents.

“Potentiating an endogenous immune response” means increasing the effectiveness or potency of an immune response in a subject. This increase in effectiveness and potency may be achieved, for example, by overcoming mechanisms that suppress the endogenous host immune response or by stimulating mechanisms that enhance the endogenous host immune response.

A “protein” refers to a chain comprising at least two consecutively linked amino acid residues, with no upper limit on the length of the chain. One or more amino acid residues in the protein may contain a modification such as, but not limited to, glycosylation, phosphorylation or disulfide bond formation. The term “protein” is used interchangeable herein with “polypeptide.”

“Selecting” or “selection,” as used herein with reference to selection of patients for treatment with anti-CTLA-4 antibodies, including next generation anti-CTLA-4 antibodies, includes any definitive step taken in the treatment protocol for a patient that dictates a future treatment step or steps. Such selection may encompass any means known in the medical arts for patient selection, including but not limited to noting in a patient's medical record, including a digital medical record, the suitability of that patient for a specific treatment regimen as opposed to alternative treatment regimens, or entering into the medical record a diagnosis that carries with it assignment of the patient to the therapeutically relevant patient subset. Such selection may also include prescription of a specific medication based on the presence of the selected characteristic, such as KRAS mutant status in their tumor, or prescribing a medication that is specifically and selectively indicated for administration to patients having the selected characteristic, such as KRAS mutant status in their tumor. A medication may be specifically and selectively indicated for administration to a subset of patients in various ways, including but not limited to articles in the medical literature suggesting such selective administration to the patient subset, inclusion on a formulary for use in the selected patient subset, treatment guidelines recommending use in the selected patient subset, insurance or government reimbursement limited to administration to patients in the subset, a statement on the drug label (package insert/prescribing information, PI) limiting use to the patient subset, or a black box warning on the drug label warning against use in patients outside the selected subset, wherein the patient subset is, e.g., patients having KRAS mutant tumors, or alternatively patients having KRAS G12C mutant tumors. “Selected” and “selection,” as used herein, do not encompass purely mental steps, and instead require an individual, such as a physician, to take an objectively verifiable action as outlined above.

A “subject” includes any human or non-human animal. The term “non-human animal” includes, but is not limited to, vertebrates such as nonhuman primates, sheep, dogs, rabbits, rodents such as mice, rats and guinea pigs, avian species such as chickens, amphibians, and reptiles. In preferred embodiments, the subject is a mammal such as a nonhuman primate, sheep, dog, cat, rabbit, ferret or rodent. Unless otherwise indicated, a subject as referred to herein is a human. The terms “subject” and “patient” are used interchangeably herein.

“Treatment” or “therapy” of a subject refers to any type of intervention or process performed on, or administration of an active agent to, the subject with the objective of reversing, alleviating, ameliorating, inhibiting, slowing down or prevent the onset, progression, development, severity or recurrence of a symptom, complication, condition or biochemical indicia associated with a disease.

### Use of Anti-CTL A-4 Antibodies to Treat KRAS Mutant Cancers

The results provided herein demonstrate that patients with KRAS mutant tumors benefit from addition of anti-CTLA-4 to their treatment regimen even when patients with wild-type KRAS tumors do not, or benefit to a lesser degree. The benefit is most easily seen in the OS data, consistent with the observation that PFS criteria may fail to fully capture therapeutic benefit in trials of immunotherapy agents. See Gyawali et al. (2018) JAMA Network Open 1:e180416. Overall Survival (OS) is the gold standard for efficacy for cancer therapy. The present invention provides methods of selectively treating patients with tumors having mutations in KRAS with anti-CTLA-4 antibodies, including next generation antibodies, and methods for selecting patients for such treatment.

Such methods of treatment directed to patients with an enhanced likelihood of benefiting from the treatment provides many advantages over treatment without segregation by KRAS status. Selective administration to the subset of likely responders within the larger patient population avoids the expense and treatment burden for patients who are unlikely to benefit from treatment. In principle, it is possible that a clinical trial that does not distinguish between KRAS mutant and KRAS WT tumors could fail to show a statistically significant result simply due to the presence of the number of non-responding KRAS WT tumors in the trial, even when the KRAS mutant population was benefiting significantly. Failure of such a trial, and the resulting denial of regulatory approval, would deny the benefit of the treatment to all patients, including many who would have benefitted.

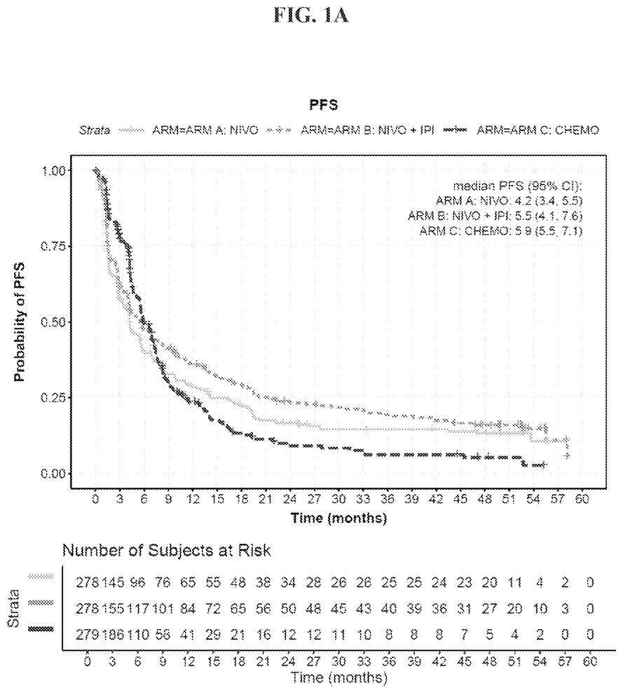

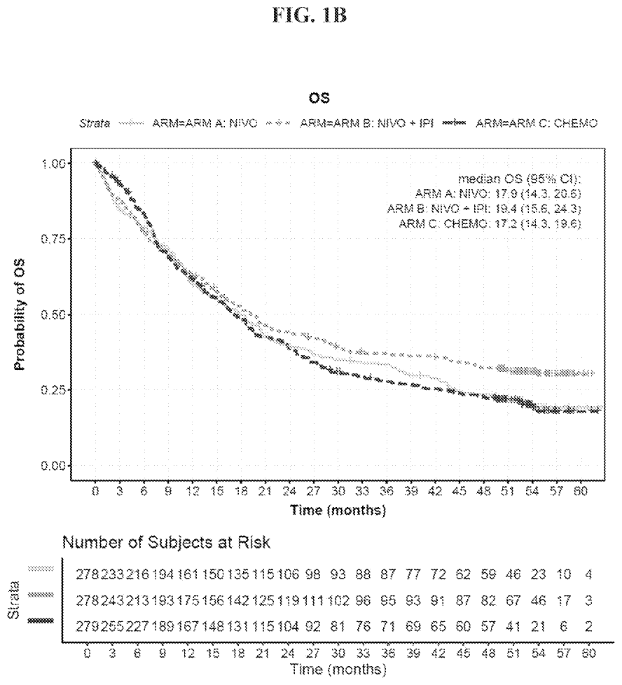

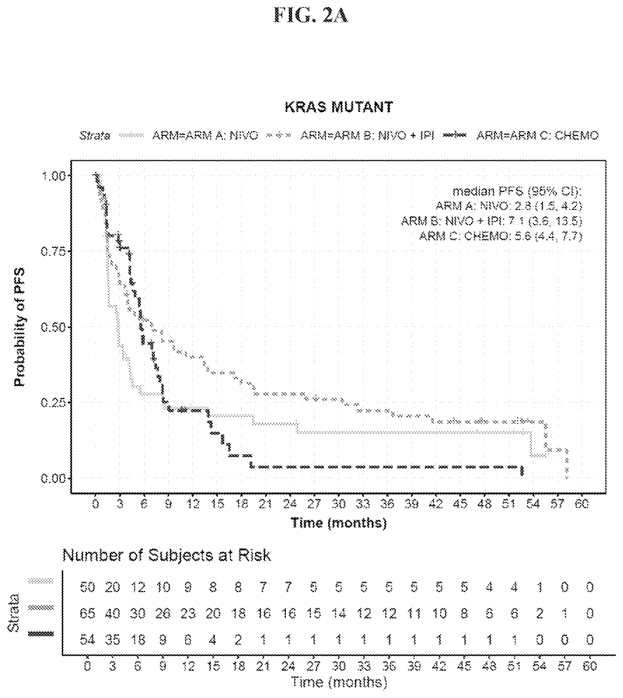

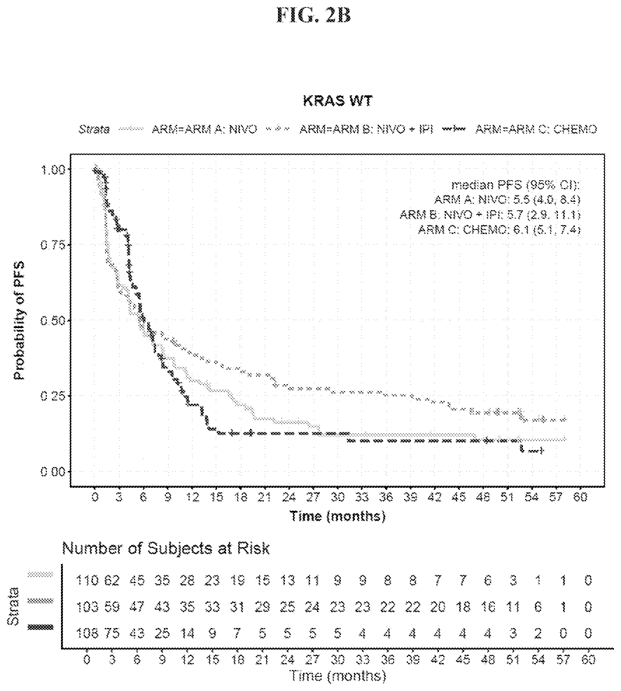

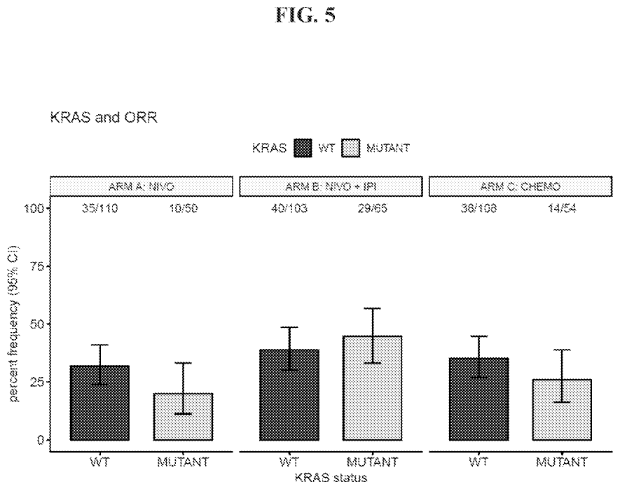

For example, FIGS. 1A and 1B show a benefit, at the PFS and OS levels respectively, of adding anti-CTLA-4 to the treatment regimen for patients regardless of KRAS status, but the beneficial effects are far more pronounced when patient data are segregated to KRAS mutation status, such as at FIGS. 2A and 2B, 3A and 3B, 4A and 4B, 5, 6A and 6B, and 7A and 7B.

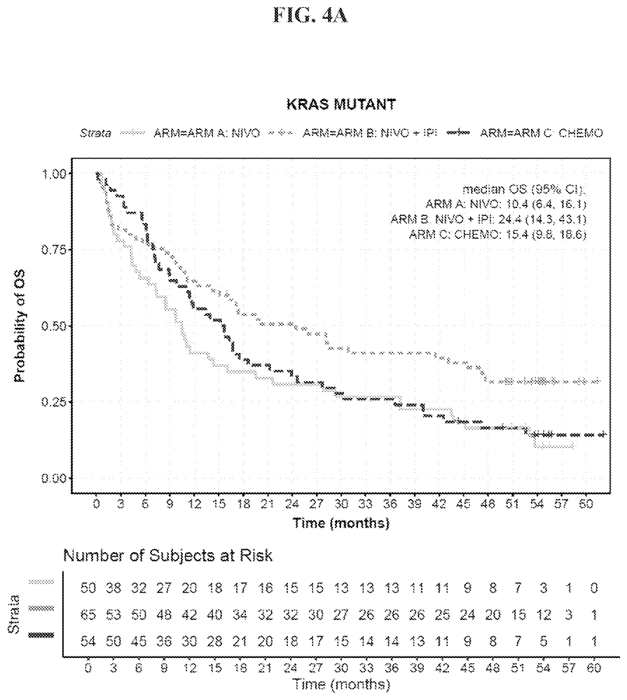

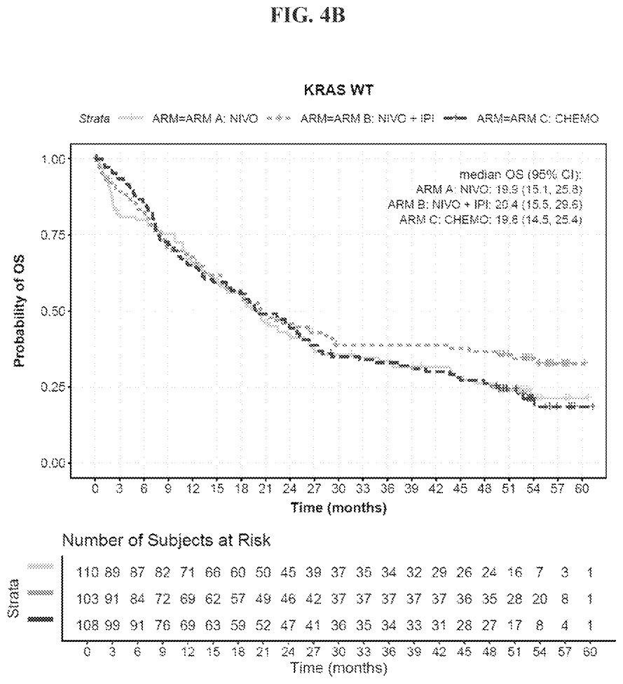

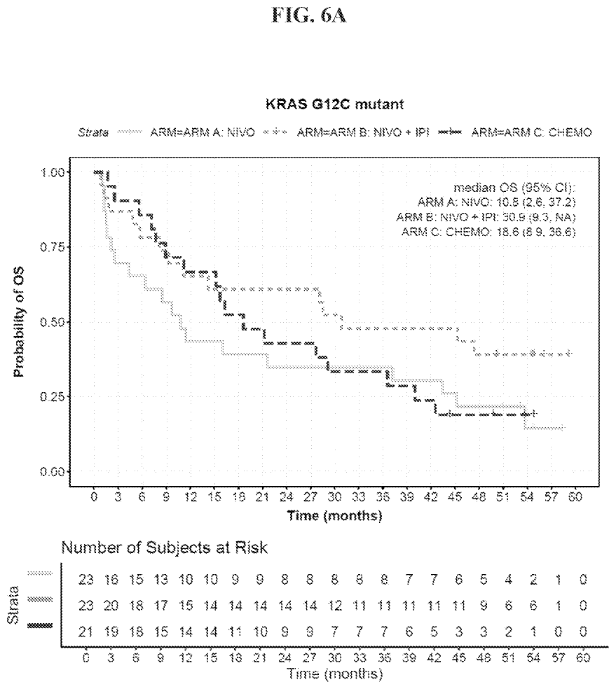

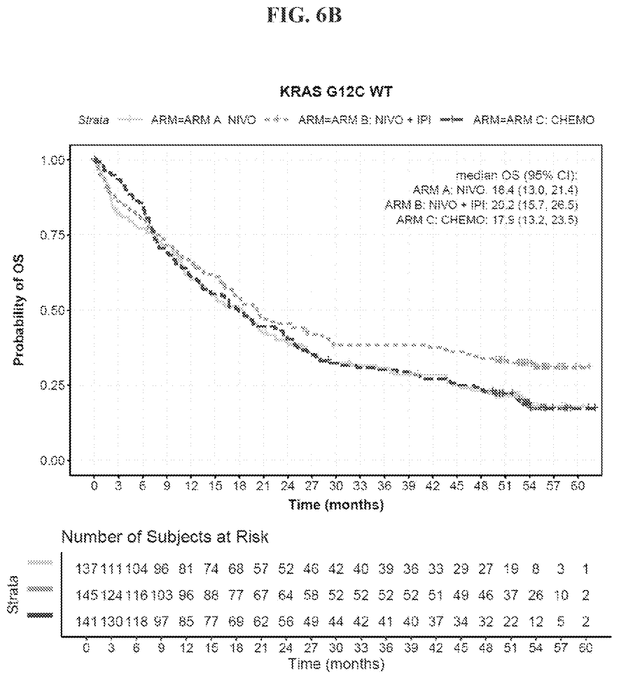

Comparison of FIGS. 2A/2B with FIGS. 3A/3B, and comparison of FIGS. 4A/4B with FIGS. 6A/6B, demonstrate that the same beneficial effect of anti-CTLA-4 treatment seen with all KRAS mutants (not sorted for the specific mutation) is also found in the KRAS G12C subset of these patients.

Anti-CTLA-4 Antibodies with Enhanced Fcy Receptor Binding

In various embodiments, anti-CTLA-4 antibodies and activatable anti-CTLA-4 antibodies for use in the methods of treatment and medical uses of the present invention are modified to exhibit enhanced Fcy receptor binding. Enhanced Fcy receptor binding is measured with reference to the Fcy receptor binding of ipilimumab. Enhanced Fcy receptor binding can be measured and quantified by assays of ADCC activity, such as the assay provided at Example 3. In various embodiments the anti-CTLA-4 antibody of the present invention exhibits 2-fold, ten-fold or greater ADCC compared with ipilimumab. In one embodiment, ADCC is measured by the NK92 cell mediated lysis assay described at Example 3. In one embodiment, the anti-CTLA-4 antibody with enhanced Fcy receptor binding of the present invention exhibits an EC50 that is at least two-fold lower than the EC50 for ipilimumab in the ADCC assay described at Example 3. In another embodiment, the anti-CTLA-4 antibody with enhanced Fcy receptor binding of the present invention exhibits an EC50 that is at least ten-fold lower than the EC50 for ipilimumab in the ADCC assay described at Example 3.

In certain embodiments the anti-CTLA-4 antibody with enhanced Fcy receptor binding comprises one or more amino acid sequence substitutions in the constant region to enhance binding to activating Fc receptors. Such antibodies may include mutations including, but not limited to, one or more of G236A, S239D, A330L and 1332E (all residue numbering per the EU numbering system). In one embodiment, the anti-CTLA-4 antibody with enhanced Fcy receptor binding comprises a human IgG1 constant domain with S239D, A330L and 1332E mutations.

In one embodiment, the anti-CTLA-4 antibody with enhanced Fcy receptor binding of the present invention is ipilimumab with reduced fucosylation, such as hypofucosylated ipilimumab or nonfucosylated ipilimumab.

### Reduced Fucosylation, Nonfucosylation and Hypofucosylation

The interaction of antibodies with FcγRs can be enhanced by modifying the glycan moiety attached to each Fc fragment at the N297 residue. In particular, the absence of core fucose residues strongly enhances ADCC via improved binding of IgG to activating FcγRIIIA (CD16) without altering antigen binding or CDC. Natsume et al. (2009) Drug Des. Devel. Ther. 3:7. There is convincing evidence that afucosylated tumor-specific antibodies translate into enhanced therapeutic activity in mouse models in vivo. Nimmerjahn & Ravetch (2005) Science 310:1510; Mossner et al. (2010) Blood 115:4393.

Modification of antibody glycosylation can be accomplished by, for example, expressing the antibody in a host cell with altered glycosylation machinery. Antibodies with reduced or eliminated fucosylation, which exhibit enhanced Fcy receptor binding, are particularly useful in the methods of the present invention. Cells with altered glycosylation machinery have been described in the art and can be used as host cells in which to express recombinant antibodies of this disclosure to thereby produce an antibody with altered glycosylation. For example, the cell lines Ms704, Ms705, and Ms709 lack the fucosyltransferase gene, FUT8 (α-(1,6) fucosyltransferase (see U.S. Pat. App. Publication No. 20040110704; Yamane-Ohnuki et al. (2004) Biotechnol. Bioeng. 87:614), such that antibodies expressed in these cell lines lack fucose on their carbohydrates. As another example, EP1176195 also describes a cell line with a functionally disrupted FUT8 gene as well as cell lines that have little or no activity for adding fucose to the N-acetylglucosamine that binds to the Fc region of the antibody, for example, the rat myeloma cell line YB2/0 (ATCC CRL 1662). PCT Publication WO 03/035835 describes a variant CHO cell line. Lec13, with reduced ability to attach fucose to Asn(297)-linked carbohydrates, also resulting in hypofucosylation of antibodies expressed in that host cell. See also Shields et al. (2002) J. Biol. Chem. 277:26733. Antibodies with a modified glycosylation profile can also be produced in chicken eggs, as described in PCT Publication No. WO 2006/089231. Alternatively, antibodies with a modified glycosylation profile can be produced in plant cells, such as Lemna. See e.g. U.S. Publication No. 2012/0276086. PCT Publication No. WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased Fcy receptor binding, and thus enhanced ADCC activity, of the antibodies. See also Umafa et al. (1999)Nat. Biotech. 17:176. Alternatively, the fucose residues of the antibody may be cleaved off using a fucosidase enzyme. For example, the enzyme alpha-L-fucosidase removes fucosyl residues from antibodies. Tarentino et al. (1975) Biochem. 14:5516. Antibodies with reduced fucosylation may also be produced in cells harboring a recombinant gene encoding an enzyme that uses GDP-6-deoxy-D-lyxo-4-hexylose as a substrate, such as GDP-6-deoxy-D-lyxo-4-hexylose reductase (RMD), as described at U.S. Pat. No. 8,642,292. Alternatively, cells may be grown in medium containing fucose analogs that block the addition of fucose residues to the N-linked glycan or a glycoprotein. U.S. Pat. No. 8,163,551; WO 09/135181.

Because nonfucosylated antibodies exhibit greatly increased biological activity due to enhanced Fcy receptor binding, compared with fucosylated antibodies, antibody preparations need not be completely free of fucosylated heavy chains to be useful in the methods of the present invention. Residual levels of fucosylated heavy chains will not significantly reduce the biological activity, such as ADCC, of a preparation of substantially nonfucosylated heavy chains. Antibodies produced in conventional CHO cells, which are fully competent to add core fucose to N-glycans, may nevertheless comprise from a few percent up to 20% nonfucosylated antibodies. Nonfucosylated antibodies may exhibit ten-fold higher affinity for CD16, and up to 30- to 100-fold enhancement of ADCC activity, so even a small increase in the proportion of nonfucosylated antibodies may drastically increase the ADCC activity of a preparation. Any preparation comprising more nonfucosylated antibodies than would be produced in normal CHO cells in culture may exhibit some level of enhanced ADCC. Such antibody preparations are referred to herein as preparations having reduced fucosylation. Depending on the original level of nonfucosylation obtained from normal CHO cells, reduced fucosylation preparations may comprise as little as 50%, 30%, 20%, 10% and even 5% nonfucosylated antibodies. Reduced fucosylation is functionally defined as preparations exhibiting two-fold or greater enhancement of ADCC compared with antibodies prepared in normal CHO cells, and not with reference to any fixed percentage of nonfucosylated species.

In other embodiments the level of nonfucosylation is structurally defined. As used herein, nonfucosylated or afucosylated (terms used synonymously) antibody preparations are antibody preparations comprising greater than 95% nonfucosylated antibody heavy chains, including 100%. Hypofucosylated antibody preparations are antibody preparations comprising less than or equal to 95% heavy chains lacking fucose, e.g. antibody preparations in which between 80 and 95% of heavy chains lack fucose, such as between 85 and 95%, and between 90 and 95%. Unless otherwise indicated, hypofucosylated refers to antibody preparations in which 80 to 95% of heavy chains lack fucose, nonfucosylated refers to antibody preparations in which over 95% of heavy chains lack fucose, and “hypofucosylated or nonfucosylated” refers to antibody preparations in which 80% or more of heavy chains lack fucose. “Fully nonfucosylated” refers to antibody preparations in which 100% of heavy chains lack fucose, and is a subset of nonfucosylated/afucosylated.

In some embodiments, hypofucosylated or nonfucosylated antibodies are produced in cells lacking an enzyme essential to fucosylation, such as FUT8 (e.g. U.S. Pat. No. 7,214,775), or in cells in which an exogenous enzyme partially depletes the pool of metabolic precursors for fucosylation (e.g. U.S. Pat. No. 8,642,292), or in cells cultured in the presence of a small molecule inhibitor of an enzyme involved in fucosylation (e.g. WO 09/135181).

The level of fucosylation in an antibody preparation may be determined by any method known in the art, including but not limited to gel electrophoresis, liquid chromatography, and mass spectrometry. Unless otherwise indicated, for the purposes of the present invention, the level of fucosylation in an antibody preparation is determined by hydrophilic interaction chromatography (or hydrophilic interaction liquid chromatography, HILIC), essentially as described at Example 4. To determine the level of fucosylation of an antibody preparation, samples are denatured, treated with PNGase F to cleave N-linked glycans, and analyzed for fucose content. LC/MS of full-length antibody chains is an alternative method to detect the level of fucosylation of an antibody preparation, but mass spectroscopy is inherently less quantitative.

### Activatable Antibodies, Such as Activatable Ipilimumab

Ipilimumab (YERVOY®) provides long-term survival in up to 25% of metastatic melanoma patients when administered at 3 mg/kg (metastatic melanoma) or 10 mg/kg (adjuvant melanoma), but treatment is often accompanied by toxicity. Activatable antibodies that are preferentially activated by tumor-associated proteases hold the promise of reducing peripheral toxicity at a given dose, allowing higher (and thus potentially more efficacious) doses for any given level of toxicity, or some intermediate trade-off of the two. Activatable Ipilimumab has been proposed as an improved, safer way to target the CTLA-4 pathway than ipilimumab, which is known to cause limiting side-effects at higher doses. WO 18/085555. Activatable Ipilimumab comprises two heavy chains and two light chains in a conventional bivalent IgG structure, albeit with additional sequence elements (including a masking moiety MM and a cleavable moiety CM) at the amino termini of the light chains. Since each CM can be cleaved independently, Activatable Ipilimumab can exist as a mixture of intact/uncleaved, mono-cleaved, and dual-cleaved forms.

Activatable antibodies have the advantage over conventional antibodies of reduced peripheral toxicity. Such reduced toxicity permits higher dosing to drive higher efficacy at the tumor site, where the antibody is selectively cleaved to a fully active form.

Exemplary embodiments and methods of the present invention are presented in the following examples.

### Example 1

An Investigational Immuno-Therapy Trial f Nivolumab, or Nivolumab Plus Ipilimumab, or Nivolumab Plus Platinum-Doublet Chemotherapy, Compared to Platinum Doublet Chemotherapy in Patients with Stage IV Non-Small Cell Lung Cancer (NSCLC)

NSCLC patients were enrolled in a clinical study called Checkmate 227 (NCT02477826: “An Investigational immuno-therapy Trial of Nivolumab, or Nivolumab Plus Ipilimumab, or Nivolumab Plus Platinum-doublet Chemotherapy, Compared to Platinum Doublet Chemotherapy in Patients with Stage IV Non-Small Cell Lung Cancer (NSCLC)”).

A first part of the study involved stage IV or recurrent NSCLC patients with no prior systemic therapy, no sensitizing EGFR mutations or known ALK alterations, no untreated NS metastases and Eastern Cooperative Oncology Group Performance Status (ECOG PS) Grade 0-1. Patients were stratified by squamous (SQ) versus non-squamous (NSQ) tumor type. Patients were further segregated based on PD-L1 expression (<1% versus ≥1% TPS) and treated with chemotherapy, nivolumab, or a combination of nivolumab and ipilimumab.

A second part of the study involved chemotherapy naïve stage IV or recurrent NSCLC patients with no EGFR/ALK mutations sensitive to available known targeted inhibitor therapy, and with ECOG PS Grade 0-1. Patients were also stratified by SQ versus NSQ tumor types. Patients were treated with histology-based platinum-based doublet chemotherapy (≤4 cycles Q3W) alone or in combination with 360 mg nivolumab (flat dose) for each cycle of therapy. Treatment with nivolumab was continued until disease progression or unacceptable toxicity, or for 2 years.

Exemplary results from Part 1 of this study are provided at FIGS. 1A, 1B, 2A, 2B, 3A, 3B, 4A, 4B, 5, 6A and 6B.

### Example 2

A Study of Nivolumab and Ipilimumab Combined with Chemotherapy Compared to Chemotherapy Alone in First Line NSCLC

NSCLC patients were enrolled in a clinical study called Checkmate 9LA (NCT03215706: “A Study of Nivolumab and Ipilimumab Combined with Chemotherapy Compared to Chemotherapy Alone in First Line NSCLC”). The study involved SQ and NSQ stage IV or recurrent NSCLC patients with no known sensitizing EGFR/ALK/ROS1 alterations and ECOG PS Grade 0-1. Patients were stratified based on PD-L1 expression (<1% versus ≥1% TPS) and by sex. Patients were treated with i) histology-based platinum-based doublet chemotherapy (4 cycles) followed by pemetrexed as maintenance (optional for NSQ), or ii) with two cycles of histology-based platinum-based doublet chemotherapy combined with 360 mg nivolumab (flat dose) Q3W and 1 mg/kg ipilimumab Q6W, followed by continuation with nivolumab and ipilimumab as maintenance for ≤24 months. Treatment of both groups continued until progression of unacceptable toxicity.

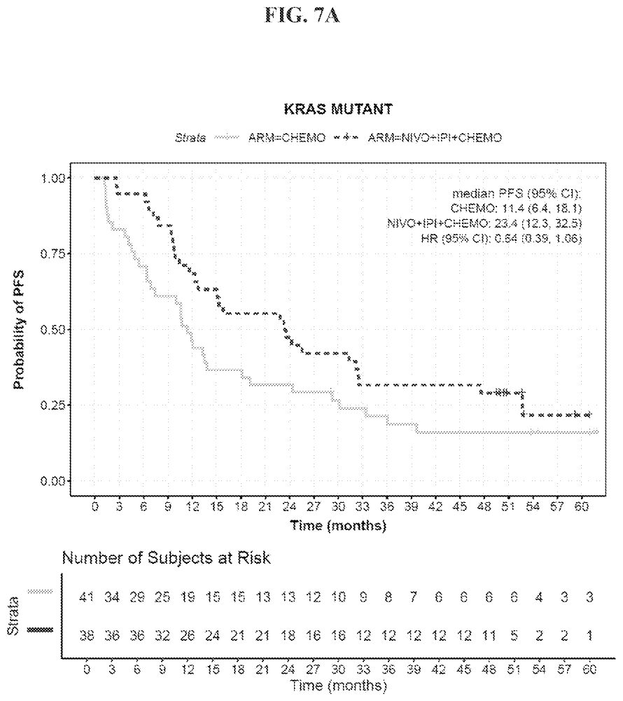

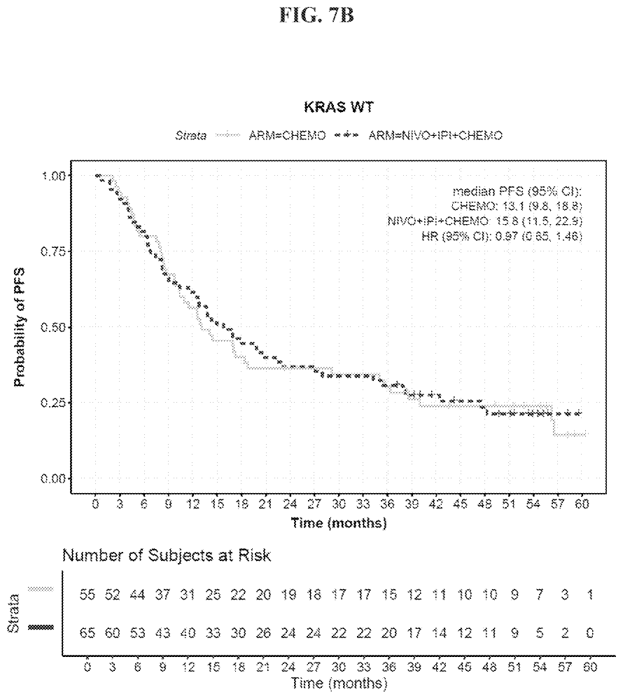

Exemplary results are provided at FIGS. 7A and 7B.

### Example 3

Anti-CTI A-4 Antibody with Enhanced ADCC Measured by Promotion of NK-mediated Cell Lysis Using Primary Human Cells

ADCC activity can be used as a measure of enhanced Fcy receptor binding of an antibody. Nonfucosylated anti-CTLA-4 antibody or activatable anti-CTLA-4 antibody is tested for its ability to promote NK cell-mediated lysis of T_{regs} c from a human donor as follows. Briefly, T_{regs} for use as target cells are separated by negative selection using magnetic beads and activated for 72 hours. NK cells for use as effectors from a human donor are separated by negative selection using magnetic beads and activated with IL-2 for 24 hrs. Calcein-labeled activated T_{regs} (Donor Leukopak AC8196) are coated with various concentrations of anti-CTLA-4 antibody or activatable anti-CTLA-4 antibody, nonfucosylated forms thereof, or an IgG1 control for 30 minutes, and then incubated with NK effector cells at a ratio of 10:1 for 2 hours. Calcein release is measured by reading the fluorescence intensity of the media using an Envision plate reader (Perkin Elmer), and the percentage of antibody-dependent cell lysis is calculated based on mean fluorescence intensity (MFI) with the following formula: [(test MFI−mean background)/(mean maximum−mean background)]×100.

### Example 4

### Assay to Determine Percentage Nonfucosylated in a Sample of Anti-CTLA-4 Antibodies

Nonfucosylated anti-CTLA-4 antibody, or activatable anti-CTLA-4 antibody, preparations are analyzed to determine the percentage of nonfucosylated heavy chains substantially as follows.

Antibodies are first denatured using urea and then reduced using DTT (dithiothreitol). Samples are then digested overnight at 37° C. with PNGase F to remove N-linked glycans. Released glycans are collected, filtered, dried, and derivatized with 2-aminobenzoic acid (2-AA) or 2-aminobenzamide (2-AB). The resulting labeled glycans are then resolved on a HILIC column and the eluted fractions are quantified by fluorescence, and dried. The fractions are then treated with exoglycosidases, such as α(1-2,3,4,6) fucosidase (BKF), which releases core α(1,6)-linked fucose residues. Untreated samples and BKF-treated samples are then analyzed by liquid chromatography. Glycans comprising α(1,6)-linked fucose residues exhibit altered elution after BKF treatment, whereas nonfucosylated glycans are unchanged. The oligosaccharide composition is also confirmed by mass spectrometry. See, e.g., Zhu et al. (2014) MAbs 6:1474.

Percent nonfucosylation is calculated as one hundred times the molar ratio of (glycans lacking a fucose α1,6-linked to the first GlcNac residue at the N-linked glycan at N297 (EU numbering) of the antibody heavy chain) to (the total of all glycans at that location (glycans lacking fucose and those having α1,6-linked fucose)).

### Example 5

### Detection of KRAS Mutation Status

The presence or absence of mutations in KRAS in tumors of human NSCLC cancer patients is determined substantially as follows. Briefly, a sample of tumor tissue is obtained, e.g. by resection or biopsy, or optionally a sample of peripheral blood is obtained (to detect circulating tumor cells). DNA can be extracted from formalin-fixed paraffin-embedded (FFPE) tissue blocks or frozen tissue. Sequence analysis can be performed by any method known in the art, but would typically involve hybridization or a polymerase chain reaction (PCR)-based method, and may employ next generation sequencing technology. Exemplary methods include PCR-based sequencing, high resolution melting analysis (HRMA), amplification refractory mutation system (ARMS) and cleavage amplification polymorphism sequence-tagged sites (PCR-RFLP). See, e.g., Tan & Du (2012) World J. Gastroenterol. 18:5171.

For example, KRAS mutation analysis in the trials described at Examples 1 and 2 was performed by complete genomic profiling using the FoundationOne® CDx test (Foundation Medicine, Cambridge Mass., USA), which employs next-generation sequencing to detect substitutions, insertion and deletion alterations (indels), and copy number alterations (CNAs) in several hundred genes and select gene rearrangements, as well as genomic signatures including microsatellite instability (MSI) and tumor mutational burden (TMB).

Subjects exhibiting mutations in KRAS may be selected for treatment using the methods of the present invention.

### Example 6

### Detection of PD-L1 Expression Status

The level of PD-L1 expression in tumors of human NSCLC patients is determined substantially as follows. Briefly, a tumor biopsy or resection specimen is obtained from the patient and the resulting tumor tissue stained for PD-L1 by immunohistochemistry. FDA-approved assays for PD-L1 expression include 22C3, 28-8, SP263, and SP142 immunoassays. See, e.g., Lanteujoul et al. (2020) J. Thoracic Oncol. 15:499. Alternatively, cytologic specimen such as bronchoalveolar lavage (BAL) wash, sputum or fine needle aspirate (FNA) may also be assayed for PD-L1 expression.

PD-L1 status may be calculated as a combined positive score (CPS) or as a tumor proportion score (TPS). PD-L1 levels reported herein are calculated as TPS scores, which reports the percentage of viable tumor cells showing partial or complete PD-L1 staining. In some embodiments of the invention, patients with tumors with TPS scores of ≥1% are considered PD-L1 positive and thus candidates for treatment with anti-CTLA-4 antibodies.

For example, PD-L1 levels in the trials described at Examples 1 and 2 were determined by immunohistochemistry (IHC) using the Dako PD-L1 PharmDx 28-8 diagnostic kit (Dako North America, Inc. (Agilent), Carpinteria Calif., USA). See Sacher & Gandhi (2016) JAMA Oncol. 2:1217. Solid tumors were considered positive for PD-L1 if there was any membrane expression of PD-L1 in tumors cells, provided at least 100 tumor cells were available for analysis.

A summary of the sequence listing is provided at Table 1.

TABLE 1
Summary of the Sequence Listing

| SEQ ID NO. | Description |
|---|---|
| 1 | human CTLA-4 (NP_005205.2) |
| 2 | human CD28 (NP_006130.1) |
| 3 | ipilimumab CDRH1 |
| 4 | ipilimumab CDRH2 |
| 5 | ipilimumab CDRH3 |
| 6 | ipilimumab CDRL1 |
| 7 | ipilimumab CDRL2 |
| 8 | ipilimumab CDRL3 |
| 9 | ipilimumab heavy chain variable domain (VH) |
| 10 | IgG1 constant domain |
| 11 | ipilimumab heavy chain lacking C-terminal K (HC) |
| 12 | ipilimumab heavy chain (HC) |
| 13 | ipilimumab light chain variable domain (VL) |
| 14 | kappa constant domain |
| 15 | ipilimumab light chain (LC) |
| 16 | Spacer QGQSGS |
| 17 | masking moiety YV39 |
| 18 | cleavable moiety 2001 |
| 19 | cleavable moiety 2011 |
| 20 | cleavable moiety 2012 |
| 21 | YV39-2001 VL |
| 22 | YV39-2011 VL |
| 23 | YV39-2012 VL |
| 24 | Spacer YV39-2011 LC |
| 25 | nivolumab heavy chain lacking C-terminal K (HC) |
| 26 | nivolumab heavy chain (HC) |
| 27 | nivolumab light chain (LC) |

With regard to antibody sequences, the Sequence Listing provides the sequences of the mature variable regions and heavy and light chains, i.e. the sequences do not include signal peptides.

### EQUIVALENTS

Those skilled in the art will recognize, or be able to ascertain using no more than routine experimentation, many equivalents of the specific embodiments disclosed herein. Such equivalents are intended to be encompassed by the following claims.

1. A method of treating cancer in a patient comprising administering an anti-CTLA-4 antibody to the patient if and only if the patient has a tumor comprising a KRAS mutation.

2. A method of treating a cancer patient having a tumor comprising:
a) detecting, or ordering the detection of, the presence or absence of one or more mutations in KRAS in the tumor; and
b) treating the cancer patient with an anti-CTLA-4 antibody if and only if the tumor comprises a mutation in KRAS.

3. A method of selecting a cancer patient having a tumor for selective treatment with an anti-CTLA-4 antibody, comprising:
a) detecting, or ordering the detection of, the presence or absence of one or more mutations in KRAS in the tumor; and
b) selecting a cancer patient for treatment with an anti-CTLA-4 antibody if and only if the tumor comprises a mutation in KRAS.

4. The method of claim 2 wherein the patient is treated with combination therapy comprising the anti-CTLA-4 antibody and a PD-1 or PD-L1 antibody.

5. The method of claim 2 wherein the patient has been previously treated with a PD-1 or PD-L1 antibody.

6. The method ofany ono of claim 2 wherein the anti-CTLA-4 antibody comprises:
a) a heavy chain comprising a heavy chain variable domain comprising:
i) a CDRH1 consisting of the sequence of SEQ ID NO: 3,
ii) a CDRH2 consisting of the sequence of SEQ ID NO: 4, and
iii) a CDRH3 consisting of the sequence of SEQ ID NO: 5;
and
b) a light chain comprising a light chain variable domain comprising:
i) a CDRL1 consisting of the sequence of SEQ ID NO: 6,
ii) a CDRL2 consisting of the sequence of SEQ ID NO: 7, and
iii) a CDRL3 consisting of the sequence of SEQ ID NO: 8.

7. The method of claim 6 wherein the anti-CTLA-4 antibody comprises:
a. a heavy chain comprising a heavy chain variable domain comprising the sequence of SEQ ID NO: 9, and
b. a light chain comprising a light chain variable domain comprising the sequence of SEQ ID NO: 13.

8. The method of claim 7 wherein the anti-CTLA-4 antibody comprises:
a. a heavy chain comprising the sequence of SEQ ID NO: 11, and
b. a light chain comprising the sequence of SEQ ID NO: 15.

9. The method of claim 8 wherein the anti-CTLA-4 antibody comprises:
a. a heavy chain comprising the sequence of SEQ ID NO: 12, and
b. a light chain comprising the sequence of SEQ ID NO: 15.

10. The method of claim 8 wherein the anti-CTLA-4 antibody is an activatable anti-CTLA-4 antibody comprising:
a. a heavy chain comprising the sequence of SEQ ID NO: 11, and
b. a light chain comprising the sequence of SEQ ID NO: 24.

11. The method of claim 10 wherein the anti-CTLA-4 antibody is an activatable anti-CTLA-4 antibody comprising:
a. a heavy chain comprising the sequence of SEQ ID NO: 12, and
b. a light chain comprising the sequence of SEQ ID NO: 24.

12. The method of claim 11 wherein the anti-CTLA-4 antibody is nonfucosylated.

13. The method of claim 12 wherein the anti-CTLA-4 antibody is fully nonfucosylated.

14. The method of claim 1 wherein the cancer is NSCLC.

15. The method of claim 14 wherein the cancer is NSQ NSCLC.

16. The method of claim 1 wherein the KRAS mutation is G12C.

17. The method of claim 1 wherein the tumor in the patient is ≥1% PD-L1.


SEQUENCE LISTING (27 sequences)

SEQ ID NO: 1 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 5 aa, synthetic construct
  REGION 1..5: note = CDR1 HC for Ipilimumab
SYTMH

SEQ ID NO: 4 - protein, 17 aa, synthetic construct
  REGION 1..17: note = CDR2 HC for Ipilimumab
FISYDGNNKYYADSVKG

SEQ ID NO: 5 - protein, 9 aa, synthetic construct
  REGION 1..9: note = CDR3 HC for Ipilimumab
TGWLGPFDY

SEQ ID NO: 6 - protein, 12 aa, synthetic construct
  REGION 1..12: note = CDR1 LC for Ipilimumab
RASQSVGSSYLA

SEQ ID NO: 7 - protein, 7 aa, synthetic construct
  REGION 1..7: note = CDR2 LC for Ipilimumab
GAFSRAT

SEQ ID NO: 8 - protein, 9 aa, synthetic construct
  REGION 1..9: note = CDR3 LC for Ipilimumab
QQYGSSPWT

SEQ ID NO: 9 - protein, 118 aa, synthetic construct
  REGION 1..118: note = Ipilimumab-VH
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 10 - protein, 329 aa, synthetic construct
  REGION 1..329: note = Human IgG1 constant HC
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 447 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 448 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 108 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 14 - protein, 107 aa, synthetic construct [sequence omitted]

SEQ ID NO: 15 - protein, 215 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 6 aa, synthetic construct
  REGION 1..6: note = Spacer
QGQSGS

SEQ ID NO: 17 - protein, 13 aa, synthetic construct
  REGION 1..13: note = Masking Moiety - YV39
CRTQLYGYNLCPY

SEQ ID NO: 18 - protein, 13 aa, synthetic construct
  REGION 1..13: note = Cleavable Moiety - 2001
ISSGLLSGRSDNH

SEQ ID NO: 19 - protein, 13 aa, synthetic construct
  REGION 1..13: note = Cleavable Moiety - 2011
ISSGLLSGRSDNP

SEQ ID NO: 20 - protein, 13 aa, synthetic construct
  REGION 1..13: note = Cleavable Moiety - 2012
ISSGLLSGRSANP

SEQ ID NO: 21 - protein, 146 aa, synthetic construct
  REGION 1..146: note = YV39-2001 VL
CRTQLYGYNLCPYGGGSSGGSISSGLLSGRSDNHGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 22 - protein, 146 aa, synthetic construct
  REGION 1..146: note = YV39-2011 VL
CRTQLYGYNLCPYGGGSSGGSISSGLLSGRSDNPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 23 - protein, 146 aa, synthetic construct
  REGION 1..146: note = YV39-2012 VL
CRTQLYGYNLCPYGGGSSGGSISSGLLSGRSANPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 24 - protein, 259 aa, synthetic construct
  REGION 1..259: note = Spacer - YV39-2011 LC
QGQSGSCRTQLYGYNLCPYGGGSSGGSISSGLLSGRSDNPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 439 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC